(19) United States
(12) Patent Application Publication
(10) Pub. No.: US 2017/0150902 A1
(43) Pub. Date: Jun. 1, 2017

(54) SYSTEMS AND METHODS FOR MEASURING RESPIRATION RATE

(71) Applicant: Draeger Medical Systems, Inc., Andover, MA (US)

(72) Inventor: Thomas Edward GAY, Manchester, NH (US)

(21) Appl. No.: 14/954,846

(22) Filed: Nov. 30, 2015

(51) Int. Cl. A61B 5/08; A61B 5/00; A61B 5/113

(52) U.S. Cl. CPC A61B 5/0816; A61B 5/113; A61B 5/7225

(57) ABSTRACT

Systems and methods for generating a signal that indicates a respiration rate of a patient are provided. Differential sinusoidal current signals having a modulation frequency are generated. The differential current signals are passed between electrodes in contact with a patient's chest. A voltage signal is received based on the passing of the differential current signals between the electrodes. The voltage signal includes a sinusoidal voltage at the modulation frequency with an amplitude that varies based on the patient's respiration. An output signal including a low-frequency component and a high-frequency component is generated by multiplying the voltage signal by a sinusoidal signal. The sinusoidal signal has the modulation frequency. The output signal is filtered to remove the high-frequency component, and the filtered output signal includes a waveform with characteristics indicative of a respiration rate of the patient. Related apparatus, systems, techniques and articles are also described.

### TECHNICAL FIELD

The subject matter described herein relates generally to respiration rate monitoring and more particularly to systems and methods for generating a signal that indicates a respiration rate of a patient.

### BACKGROUND

Respiration rate is the number of breaths a person takes per minute. Respiration rates can increase or decrease with fever, illness, and other medical conditions, and thus, a patient's respiration rate is frequently monitored as a means of analyzing the patient's medical state of health. Respiration rate can be represented as a number of breaths per minute or as a frequency (e.g., a frequency of 1 Hz corresponds to 60 breaths per minute). A patient's respiration rate can be measured manually (e.g., by having a clinician count the number of breaths that the patient takes over a period of time) or via automated respiration measurement systems.

### SUMMARY

Systems and methods for generating a signal that indicates a respiration rate of a patient are provided. In one aspect, sinusoidal differential current signals having a modulation frequency are generated. The differential current signals are passed between electrodes in contact with a patient's chest. A voltage signal is received based on the passing of the differential current signals between the electrodes. The voltage signal includes a sinusoidal voltage at the modulation frequency with an amplitude that varies based on the patient's respiration. An output signal including a low-frequency component and a high-frequency component is generated by multiplying the voltage signal by a sinusoidal signal. The sinusoidal signal has the modulation frequency and a constant amplitude. The output signal is filtered to remove the high-frequency component, and the filtered output signal includes a waveform with characteristics indicative of a respiration rate of the patient.

In another interrelated aspect for generating a signal that indicates a respiration rate of a patient, an amplitude-modulated (AM) signal with amplitude that varies based on a patient's respiration is received. The AM signal is based on a passing of a sinusoidal current signal between electrodes in contact with the patient's chest. The AM signal is demodulated using a multiplier. An output of the multiplier includes a waveform with characteristics indicative of a respiration rate of the patient.

In a further interrelated aspect, a system for generating a signal that indicates a respiration rate of a patient includes a current source configured to generate sinusoidal differential current signals having a modulation frequency. The differential current signals are passed between electrodes in contact with a patient's chest to generate a voltage signal. The voltage signal is a sinusoidal voltage at the modulation frequency with an amplitude that varies based on the patient's respiration. A signal source is configured to generate a sinusoidal signal having the modulation frequency and a constant amplitude. A multiplier is configured to generate an output signal including a low-frequency component and a high-frequency component by multiplying the voltage signal by the sinusoidal signal. A filter is configured to filter the output signal to remove the high-frequency component. The filtered output signal includes a waveform with characteristics indicative of a respiration rate of the patient.

In a further interrelated aspect, a patient monitoring device includes a system for generating a signal that indicates a respiration rate of a patient. The system for generating the signal includes a current source configured to generate sinusoidal differential current signals having a modulation frequency. The differential current signals are passed between electrodes in contact with a patient's chest to generate a voltage signal. The voltage signal is a sinusoidal voltage at the modulation frequency with an amplitude that varies based on the patient's respiration. A signal source is configured to generate a sinusoidal signal having the modulation frequency and a constant amplitude. A multiplier is configured to generate an output signal including a low-frequency component and a high-frequency component by multiplying the voltage signal by the sinusoidal signal. A filter is configured to filter the output signal to remove the high-frequency component. The filtered output signal includes a waveform with characteristics indicative of the respiration rate of the patient.

The subject matter described herein provides many technical advantages. As described below, a multiplier is used to demodulate a respiration signal. The use of the multiplier eliminates sources of noise that are present in conventional systems, thus enabling respiration readings of higher accuracy. In addition, this noise reduction will yield better performance for both ECG and Pacer pulse detection functions. The systems and methods described herein also eliminate complexities of differential signal processing present in the conventional systems and enable signal processing via a simplified filter design. These technical advantages and others are described in detail below.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a block diagram depicting an example system for generating a signal indicative of a respiration rate of a patient;

FIG. 2 is a schematic of an example system for measuring a patient's respiration rate;

FIG. 3 is a flowchart depicting example steps of a method for generating a signal that indicates a respiration rate of a patient; and

FIG. 4 depicts an example patient monitoring device.

### DETAILED DESCRIPTION

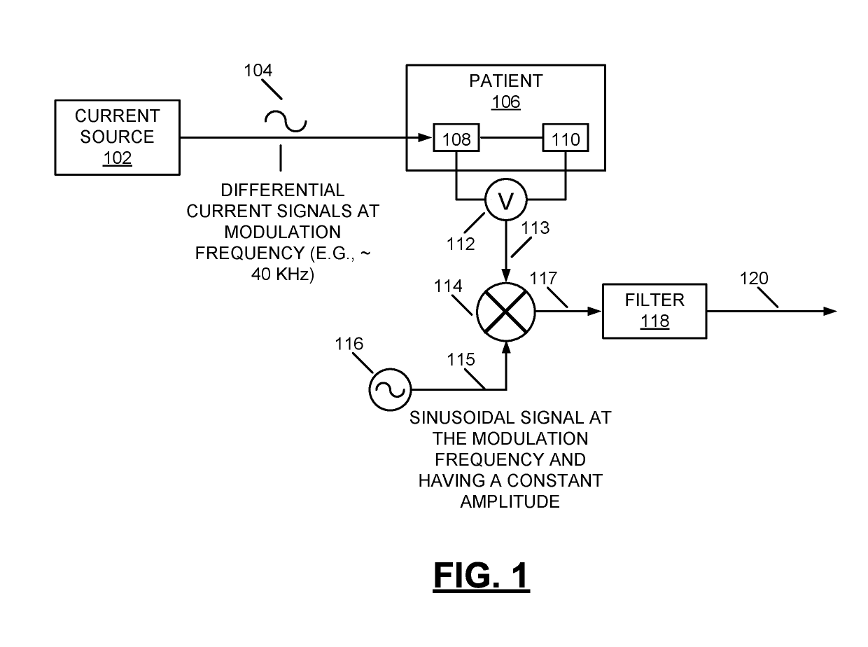

FIG. 1 is a block diagram depicting an example system for generating a signal indicative of a respiration rate of a patient 106. The system includes a current source 102 configured to generate differential current signals 104. Each of the differential current signals 104 is a sinusoidal current having a modulation frequency and a constant amplitude. The modulation frequency is a relatively high frequency that is one or more orders of magnitude higher than a frequency representation of the patient's respiration rate. For example, a typical respiration rate for a healthy adult at rest is 12-20 breaths per minute. Represented as frequencies, such respiration rates correspond to a frequency range of 0.20-0.33 Hz. By contrast, in examples, the differential sinusoidal current signals 104 have a frequency of approximately 40 KHz.

As illustrated in FIG. 1, the differential sinusoidal current signals 104 are passed between electrodes 108, 110 that are in contact with a chest of the patient 106. A voltage between the electrodes 108, 110 is proportional to an impedance of the patient's chest and varies as a function of respiration as the patient's chest expands and contracts. A voltage sensing circuit 112 reads the voltage between the electrodes 108, 110 and generates a corresponding voltage signal 113. Because the differential current signals 104 are sinusoidal signals having the modulation frequency, the voltage signal 113 is a sinusoidal voltage at the modulation frequency with an amplitude that varies based on the patient's respiration. Thus, the voltage signal 113 is an amplitude-modulated (AM) sinusoidal signal.

As noted above, the voltage signal 113 is an AM signal with an amplitude that varies based on the patient's respiration. To demodulate this AM signal, a multiplier 114 is utilized. As shown in FIG. 1, the multiplier 114 receives the voltage signal 113 from the voltage sensing circuit 112. The multiplier 114 also receives a sinusoidal signal 115 having the modulation frequency (e.g., approximately 40 KHz) and a constant amplitude from a signal source 116. The sinusoidal signal 115 can be a voltage signal having the constant amplitude. As noted above, the differential sinusoidal current signals 104 also have a constant amplitude, and in examples, the constant amplitude of the sinusoidal voltage signal 115 is proportional to that of the differential sinusoidal current signals 104.

The multiplier 114 demodulates the voltage signal 113 by multiplying the voltage signal 113 by the sinusoidal signal 115. As noted above, the signals 113 and 115 share the same modulation frequency, but the AM voltage signal 113 carries additional information about the patient's respiration rate via its varying amplitude. Multiplying the signals 113, 115 at the multiplier 114 enables this additional information to be extracted from the signal 113. Specifically, when the signals 113, 115 are multiplied, a resulting output signal 117 of the multiplier 114 includes a high-frequency component and a low-frequency component. The high-frequency component has a frequency that is double the modulation frequency. The low-frequency component includes a waveform with an amplitude that varies based on the patient's respiration rate. This waveform has a relatively low frequency that is one or more orders of magnitude lower than the frequency of the high-frequency component. In an example, the high-frequency component has a frequency of approximately 80 KHz, and the low-frequency component has a frequency in the range of 0.25-3.5 Hz, depending on the patient's respiration rate.

To remove the high-frequency component from the output signal 117, a filter 118 is used. The filter 118 is a low-pass filter, in examples. A filtered output signal 120 generated by the filter 118 retains the low-frequency component of the output signal 117, which includes the waveform with an amplitude that varies based on the patient's respiration rate. In some examples, additional filtering is performed on the output signal 117 to generate the filtered output signal 120 (e.g., filtering to remove a direct current (DC) bias from the output signal 117, etc.). The filtered output signal 120 can be received by various components (e.g., analog or digital signal processing systems, etc.), and the respiration rate of the patient 106 can be calculated based on the signal 120. Data characterizing the calculated respiration rate can be stored in a memory, displayed via a display device, and/or transmitted to a remote computing system, among other possibilities.

The systems and methods described herein for measuring a respiration rate of a patient differ from conventional approaches. For example, conventional systems for measuring a patient's respiration rate make use of multiple sets of switches. A modulation set of switches is used in generating a bias current, and a second set of switches is used in sampling waveforms from electrodes in contact with the patient. The switches are opened and closed at high speeds to achieve these purposes, and the switching of the two sets must be precisely timed relative to each other. An example system that uses this conventional arrangement is Texas Instruments' ADS1298R, which is known to those of ordinary skill in the art. The conventional systems utilizing these switches commonly suffer from timing issues due to the aforementioned timing requirements. Further, the high-speed switching used in the conventional systems creates noise that can corrupt signals used in these systems (e.g., signals representing respiration rate, electrocardiogram (ECG) signals, pacemaker signals, etc.). The conventional systems also rely on complex, sampled data differential signal processing techniques.

In contrast to these conventional approaches, the systems and methods described herein do not utilize high-speed switching. The systems and methods described herein are thus not susceptible to the noise issues associated with high-speed switching and can have respiration readings of higher accuracy than the conventional systems. The approaches of the subject matter described herein also eliminate the complexities of differential switched signal processing present in the conventional systems. It is further noted that the instant disclosure's use of a multiplier for demodulating a respiration signal is in contrast to the conventional systems, which do not utilize this technique. Additional differences between the conventional systems and the subject matter described herein are detailed throughout this disclosure.

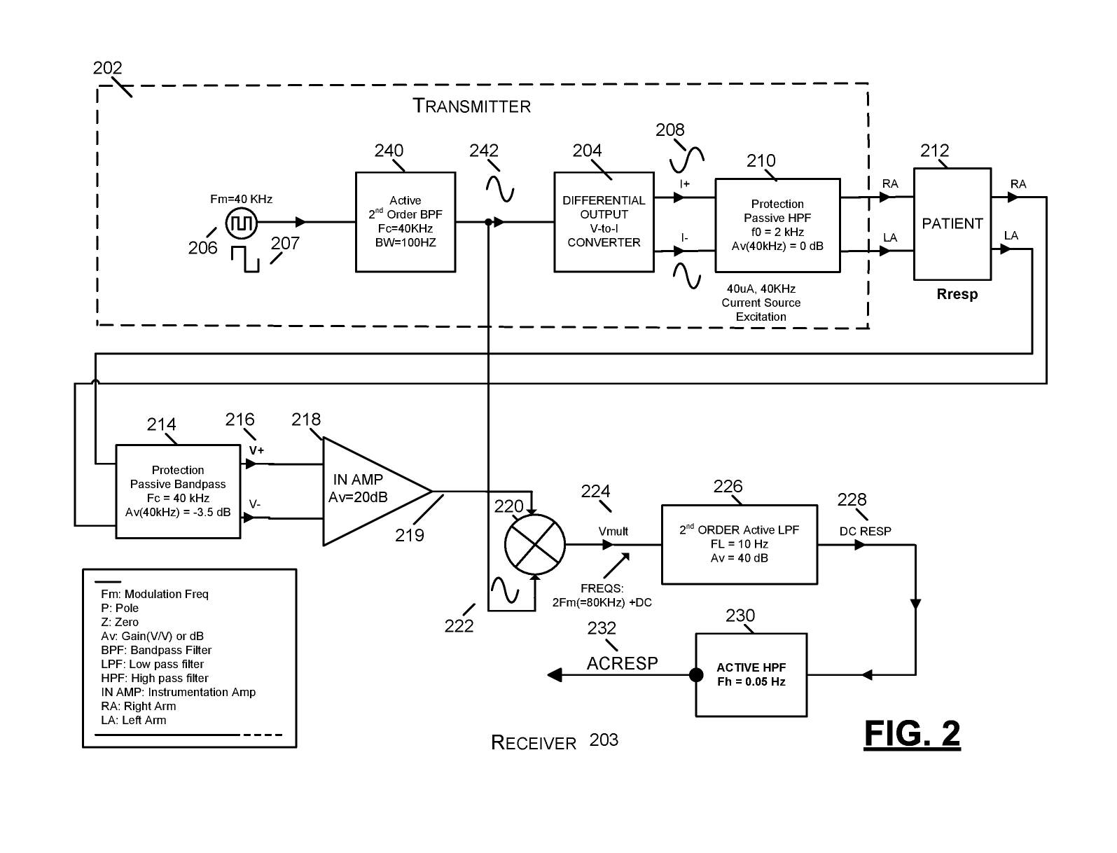

FIG. 2 is a schematic of an example system for measuring a respiration rate of a patient 212. The system of FIG. 2 is similar to the system of FIG. 1 but depicts additional details not shown in FIG. 1. As illustrated in FIG. 2, the system includes a transmitter 202 and a receiver 203. The transmitter 202 is used in generating current signals that are passed between electrodes in contact with the patient's chest, and the receiver 203 is used to read voltage signals at the electrodes and generate an output signal indicative of the patient's respiration rate. The transmitter 202 includes a signal source 206 that is configured to generate a digital clock signal 207 having a modulation frequency equal to approximately 40 KHz (e.g., 39.2 KHz). In examples, the digital clock signal 207 is a voltage signal including square waves or rectangular waves.

The frequencies noted herein with reference to FIG. 2 are examples only, and other frequencies are used in other examples. For instance, although the modulation frequency of the digital clock signal 207 is indicated as being approximately 40 KHz in the example of FIG. 2, in other examples, the modulation frequency is equal to 10 KHz, 20 KHz, 30 KHz, 50 KHz, or another frequency. The modulation frequency of the digital clock signal 207 is a relatively high frequency that is one or more orders of magnitude higher than a frequency representation of the patient's respiration rate. As noted above, the patient's respiration rate generally corresponds to a frequency range of 0.20-0.33 Hz, and the modulation frequency of the digital clock signal 207 can be approximately 40 KHz or a different frequency (e.g., a different frequency in the kilohertz range).

The digital clock signal 207 is received at an active, second-order band-pass filter 240. In some embodiments, a filter that is described herein as active can be passive, and vice versa. As indicated in the figure, the band-pass filter 240 has a center frequency equal to the modulation frequency of the digital clock signal 207 (e.g., approximately 40 KHz) and a passband of approximately 100 Hz. The band-pass filter 240 converts the square or rectangular waves of the digital clock signal 207 into sinusoidal waves. This is shown in FIG. 2, which depicts the band-pass filter 240 outputting a sinusoidal signal 242. The use of sinusoidal signals in the system of FIG. 2 eliminates harmonic content present in signals with square or rectangular waves, which can be a source of noise. The sinusoidal signal 242 is received at a differential output voltage-to-current converter 204, which converts the voltage levels of the sinusoidal signal 242 into differential sinusoidal current signals. Specifically, in the example of FIG. 2, the voltage-to-current converter 204 generates a differential pair 208 of sinusoidal current signals that are 180 degrees out of phase with each other. Both of the sinusoidal current signals of the differential pair 208 have the modulation frequency of approximately 40 KHz, and both signals are constant amplitude signals that share a same amplitude. The current signals of the differential pair 208 have a magnitude of 40 μA in the example of FIG. 2. In other examples, current signals of other magnitudes (e.g., 30 μA, 50 μA, 60 μA, etc.) are used.

The differential pair 208 of current signals are filtered at a passive high-pass filter 210 having a cutoff frequency of 2 KHz in the example of FIG. 2. The high-pass filter 210 can include a passive network including resistors and capacitors and/or other components. The high-pass filter 210 removes low-frequency components from the current signals prior to passing the current signals between electrodes in contact with a chest of the patient 212. These electrodes are denoted as RA (“right-arm”) and LA (“left-arm”) in the figure to indicate the different sides of the patient's chest on which the electrodes are placed.

Voltages across the electrodes are a result of the current passing through the patient's body, as described above with reference to FIG. 1. Such voltages are filtered at a band-pass filter 214 having a center frequency equal to the modulation frequency of approximately 40 KHz and a passband of approximately 100 Hz. The band-pass filter 214 filters the voltages to remove high- and low-frequency components outside of the filter's passband and provides protection to the system (e.g., in the event of defibrillation). Like the high-pass filter 210 described above, the band-pass filter 214 can include a passive network of resistors and capacitors and/or other components. The band-pass filter 214 outputs first and second voltage signals 216, and these voltage signals 216 are received at an instrumentation amplifier 218. The voltage signals 216 (labeled V+ and V− in FIG. 2) are a result of the passing of the differential pair 208 of current signals through the patient's body, with each of the two voltage signals 216 corresponding to a respective current signal of the differential pair 208. Thus, for example, the V+ voltage signal is based on the passing of the I+ current signal between the electrodes, and the V− voltage signal is based on the passing of the I− current signal between the electrodes.

The V+ and V− voltage signals 216 are sinusoidal voltages having the modulation frequency of approximately 40 KHz. Each of these signals 216 has an amplitude that varies based on the patient's respiration. The amplitude variation is a result of the patient's chest expanding and contracting as he or she breathes, with the expansion and contraction causing the impedance of the patient's chest to vary, as described above. The instrumentation amplifier 218 is a precise, high-impedance, differential amplifier that is configured to (i) take a difference between the V+ and V− signals 216, and (ii) amplify the difference. An output of the instrumentation amplifier 218 is a single-ended voltage signal 219. The single-ended voltage signal 219 is a sinusoidal voltage at the modulation frequency of approximately 40 KHz with an amplitude that varies based on the patient's respiration. Thus, the voltage signal 219 is an amplitude-modulated (AM) signal, similar to the voltage signal 113 described above with reference to FIG. 1.

To demodulate the single-ended voltage signal 219, this signal is provided to a multiplier 220. In examples, the multiplier 220 is a four-quadrant analog multiplier (e.g., Analog Devices AD534) and can include an integrated circuit and/or other components. In other examples, the multiplier 220 is a two-quadrant multiplier. Further, in some embodiments, a non-analog (i.e., digital) multiplier is used. The multiplier 220 also receives a sinusoidal signal 222 having the modulation frequency of approximately 40 KHz and a constant amplitude. In an example, the sinusoidal signal 222 is a voltage signal having the constant amplitude. As noted above, the sinusoidal current signals of the differential pair 208 have a constant amplitude that is the same in both signals, and in examples, the constant amplitude of the sinusoidal voltage signal 222 is proportional to that of the differential pair 208. Although the example of FIG. 2 shows the sinusoidal signal 222 being received at the multiplier 220 from the band-pass filter 240, in other examples, the sinusoidal signal is received from a different component (e.g., a signal source that is separate from the component 240, etc.).

The multiplier 220 demodulates the single-ended voltage signal 219 by multiplying the voltage signal 219 by the sinusoidal signal 222. As noted above, the single-ended voltage signal 219 and the sinusoidal signal 222 share the same modulation frequency of approximately 40 KHz, but the single-ended voltage signal 219 carries additional information about the patient's respiration rate via its varying amplitude. Multiplying the signals 219, 222 at the multiplier 220 enables this additional information to be extracted from the signal 219. When the signals 219, 222 are multiplied, a resulting Vmult output signal 224 of the multiplier 220 includes a high-frequency component and a low-frequency component.

The high-frequency component of the Vmult output signal 224 has a frequency that is double the modulation frequency and is thus equal to approximately 80 KHz (e.g., 78.4 KHz) in the example of FIG. 2. The low-frequency component of the Vmult output signal 224 includes a waveform with an amplitude that varies based on the patient's respiration rate. This waveform has a relatively low frequency that is one or more orders of magnitude lower than the approximately 80 KHz frequency of the high-frequency component. In an example, the low-frequency component has a frequency in the range of 0.25-3.5 Hz, depending on the patient's respiration rate. The low-frequency component can also include a DC bias that depends on various factors, including characteristics of the patient, characteristics of the electrodes and their placement on the patient's chest, and/or other factors.

To remove the high-frequency component from the Vmult output signal 224, a second-order, active low-pass filter 226 having a cutoff frequency of 10 Hz is used. As noted above, the low-frequency component has a frequency in the range of 0.25-3.5 Hz, such that the low-frequency component passes through the filter 226 while the high-frequency component at approximately 80 KHz is removed. The large frequency differential between the low-frequency component and the high-frequency component enables a relatively simple filter design. A filtered output signal DC_RESP 228 generated by the filter 226 retains the low-frequency component of the Vmult output signal 224, which includes the waveform with an amplitude that varies based on the patient's respiration rate.

As noted above, the low-frequency component of the Vmult output signal 224 can include a DC bias, and the filtering by the low-pass filter 226 does not remove this DC bias. To remove the DC bias from the filtered output signal DC_RESP 228, this signal is received at an active high-pass filter 230 having a cutoff frequency of 0.05 Hz in the example of FIG. 2. The high-pass filter 230 removes the DC bias from the signal DC_RESP 228 while retaining the low-frequency component in the range of 0.25-3.5 Hz. An ACRESP output signal 232 of the high-pass filter 230 is a waveform with an amplitude that varies based on the patient's respiration rate. The ACRESP output signal 232 can be received by various components (e.g., analog or digital signal processing systems, etc.), and the respiration rate of the patient 212 can be calculated based on the signal 232. In some examples, the ACRESP output signal 232 is received at an analog-to-digital converter (ADC), and a resulting digital signal output by the ADC is processed to determine the respiration rate of the patient. Although the system of FIG. 2 is described herein as utilizing analog signal processing, in some embodiments, at least some of the signal processing can be non-analog (i.e., digital). Data characterizing the respiration rate can be displayed on a display device, stored in a memory, and/or transmitted to a remote computing system, among other possibilities.

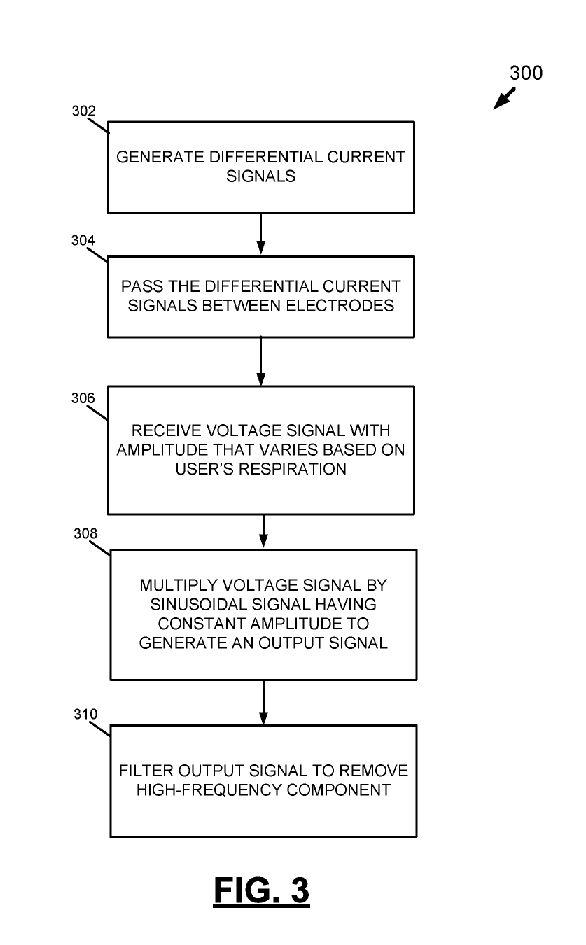

FIG. 3 is a flowchart 300 depicting example steps of a method for generating a signal that indicates a respiration rate of a patient. At 302, differential sinusoidal current signals having a modulation frequency are generated. At 304, the differential current signals are passed between electrodes in contact with a patient's chest. At 306, a voltage signal is received based on the passing of the differential current signals between the electrodes. The voltage signal includes a sinusoidal voltage at the modulation frequency with an amplitude that varies based on the patient's respiration. At 308, an output signal including a low-frequency component and a high-frequency component is generated by multiplying the voltage signal by a sinusoidal signal. The sinusoidal signal has the modulation frequency and a constant amplitude. At 310, the output signal is filtered to remove the high-frequency component, and the filtered output signal includes a waveform with characteristics indicative of a respiration rate of the patient.

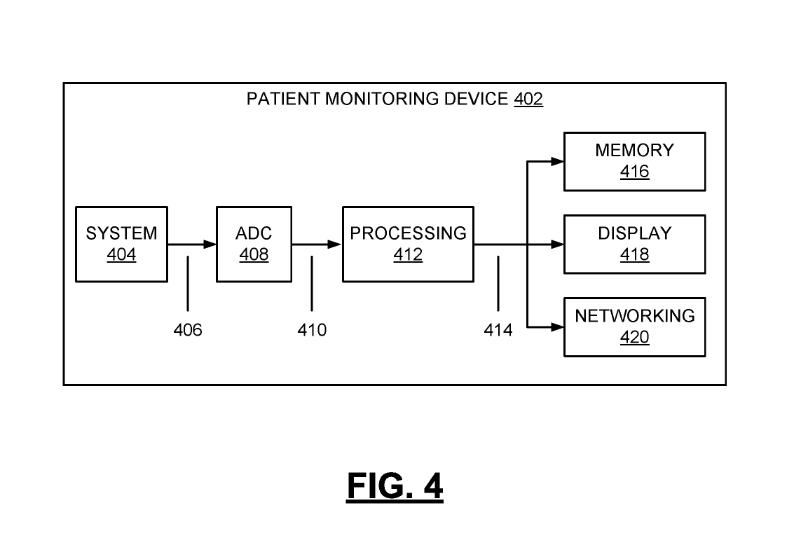

FIG. 4 depicts an example patient monitoring device 402. The patient monitoring device 402 includes a system 404 for generating a signal that indicates a respiration rate of a patient. In examples, the system 404 is (i) the system described above with reference to FIG. 1, (ii) the system described above with reference to FIG. 2, or (iii) another system utilizing the approaches described herein for generating a signal that indicates a respiration rate of a patient. The system 404 generates an output signal 406 with characteristics indicative of the respiration rate of the patient. In the example of FIG. 4, the output signal 406 is an analog signal that is received at an ADC 408. The ADC 408 is configured to generate a digital signal 410 based on the analog output signal 406. The digital signal 410 is received at a processing module 412 that is configured to process the digital signal 410 to determine the patient's respiration rate. The processing module 412 is implemented via a microprocessor, microcontroller, system on a chip (SOC), or other fixed or programmable logic, in examples, and may include one or more processors or processor cores.

Data 414 characterizing the respiration rate, as determined using the processing module 412, is stored in a memory 416 of the patient monitoring device 402, in examples. The data 414 is transmitted to a display 418 of the patient monitoring device 402, in examples, thus enabling the data 414 to be displayed at the device 402. Further, in some examples, the data 414 is received at a networking component 420 of the patient monitoring device 402. The networking component 420 is used in transmitting the data 414 to another system (e.g., a computing system accessible via a wired or wireless network). The patient monitoring device 402 of FIG. 4 is only an example, and in other examples, a patient monitoring device includes a different set of components (e.g., the patient monitoring device may not include all of the components shown in FIG. 4, and/or the patient monitoring device may include additional other components not shown in FIG. 4).

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method for generating a signal that indicates a respiration rate of a patient, the method comprising:
generating differential current signals that each comprise a sinusoidal current having a modulation frequency;
passing the differential current signals between electrodes in contact with a patient's chest;
receiving a voltage signal based on the passing of the differential current signals between the electrodes, the voltage signal comprising a sinusoidal voltage at the modulation frequency with an amplitude that varies based on the patient's respiration;
generating an output signal including a low-frequency component and a high-frequency component by multiplying the voltage signal by a sinusoidal signal having the modulation frequency and a constant amplitude; and
filtering the output signal to remove the high-frequency component, the filtered output signal comprising a waveform with characteristics indicative of a respiration rate of the patient.

2. The method of claim 1, further comprising:
calculating the respiration rate of the patient based on the filtered output signal; and
providing data characterizing the respiration rate.

3. The method of claim 2, wherein the providing of the data characterizing the respiration rate comprises:
storing the data;
loading the data into a memory;
displaying the data; or
transmitting the data to a remote computing system.

4. The method of claim 1, wherein the differential current signals comprise a differential pair of signals 180 degrees out of phase with each other, and the voltage signal comprises a single-ended voltage signal, wherein generating the single-ended voltage signal comprises:
receiving first and second voltage signals based on the passing of the differential pair of signals between the electrodes, each of the first and second voltage signals corresponding to a respective signal of the differential pair, and the first and second voltage signals comprising sinusoidal voltages at the modulation frequency with amplitudes that vary based on the patient's respiration; and
taking a difference between the first and second voltage signals to generate the single-ended voltage signal.

5. The method of claim 1, wherein the respiration rate of the patient, represented as a frequency, is one or more orders of magnitude lower than the modulation frequency.

6. The method of claim 1, wherein the sinusoidal signal is a voltage signal having the constant amplitude, and wherein the sinusoidal current signals have a second constant amplitude, the constant amplitude of the voltage signal being proportional to the second constant amplitude of the sinusoidal current signals.

7. The method of claim 1, further comprising:
performing additional filtering on the filtered output signal to remove a direct current (DC) bias from the filtered output signal.

8. The method of claim 1, wherein a frequency of the low-frequency component is one or more orders of magnitude lower than a frequency of the high-frequency component.

9. A method for generating a signal that indicates a respiration rate of a patient, the method comprising:
receiving an amplitude-modulated (AM) signal with amplitude that varies based on a patient's respiration, the AM signal being based on a passing of a sinusoidal current signal between electrodes in contact with the patient's chest; and
demodulating the AM signal using a multiplier, an output of the multiplier comprising a waveform with characteristics indicative of a respiration rate of the patient.

10. The method of claim 9, wherein the sinusoidal current signal and the AM signal have a same frequency, and wherein the demodulating of the AM signal comprises: multiplying, at the multiplier, the AM signal by a sinusoidal signal having the frequency and a constant amplitude.

11. The method of claim 9, further comprising:
calculating the respiration rate of the patient based on the output of the multiplier; and
providing data characterizing the respiration rate.

12. The method of claim 11, wherein the providing of the data characterizing the respiration rate comprises:
storing the data in a memory;
displaying the data; or
transmitting the data to a remote computing system.

13. A system for generating a signal that indicates a respiration rate of a patient, the system comprising:
a current source configured to generate differential current signals that each comprise a sinusoidal current having a modulation frequency, the differential current signals being passed between electrodes in contact with a patient's chest to generate a voltage signal, the voltage signal comprising a sinusoidal voltage at the modulation frequency with an amplitude that varies based on the patient's respiration;
a signal source configured to generate a sinusoidal signal having the modulation frequency and a constant amplitude;
a multiplier configured to generate an output signal including a low-frequency component and a high-frequency component by multiplying the voltage signal by the sinusoidal signal; and
a filter configured to filter the output signal to remove the high-frequency component, the filtered output signal comprising a waveform with characteristics indicative of a respiration rate of the patient.

14. The system of claim 13, wherein the multiplier comprises a four-quadrant multiplier.

15. The system of claim 13, wherein the multiplier comprises a two-quadrant multiplier.

16. The system of claim 13, wherein the differential current signals comprise a differential pair of signals 180 degrees out of phase with each other, and the voltage signal comprises a single-ended voltage signal, the system further comprising:
a differential amplifier configured to receive first and second voltage signals based on the passing of the differential pair of signals between the electrodes, each of the first and second voltage signals corresponding to a respective signal of the differential pair, and the first and second voltage signals comprising sinusoidal voltages at the modulation frequency with amplitudes that vary based on the patient's respiration, wherein the differential amplifier generates the single-ended voltage signal by taking a difference between the first and second voltage signals.

17. The system of claim 16, wherein the differential amplifier comprises an instrumentation amplifier.

18. The system of claim 13, wherein the respiration rate of the patient, represented as a frequency, is one or more orders of magnitude lower than the modulation frequency.

19. The system of claim 13, wherein the sinusoidal signal is a voltage signal having the constant amplitude, and wherein the sinusoidal current signals have a second constant amplitude, the constant amplitude of the voltage signal being proportional to the second constant amplitude of the sinusoidal current signals.

20. The system of claim 13, further comprising:
a second filter configured to perform additional filtering on the filtered output signal to remove a direct current (DC) bias from the filtered output signal.

21. The system of claim 13, wherein a frequency of the low-frequency component is one or more orders of magnitude lower than a frequency of the high-frequency component.

22. A patient monitoring device comprising:
a system for generating a signal that indicates a respiration rate of a patient, the system including:
a current source configured to generate differential current signals that each comprise a sinusoidal current having a modulation frequency, the differential current signals being passed between electrodes in contact with a patient's chest to generate a voltage signal, the voltage signal comprising a sinusoidal voltage at the modulation frequency with an amplitude that varies based on the patient's respiration,
a signal source configured to generate a sinusoidal signal having the modulation frequency and a constant amplitude,
a multiplier configured to generate an output signal including a low-frequency component and a high-frequency component by multiplying the voltage signal by the sinusoidal signal, and
a filter configured to filter the output signal to remove the high-frequency component, the filtered output signal comprising a waveform with characteristics indicative of the respiration rate of the patient.

23. The patient monitoring device of claim 22, further comprising:
an analog-to-digital converter (ADC) configured to generate a digital signal based on the filtered output signal; and
a processing module configured to process the digital signal to determine the respiration rate.

24. The patient monitoring device of claim 22, further comprising:
a memory configured to store data characterizing the respiration rate.

25. The patient monitoring device of claim 22, further comprising:
a display configured to display data characterizing the respiration rate.
